(19) United States
(12) Patent Application Publication
(10) Pub. No.: US 2026/0174730 A1
(43) Pub. Date: Jun. 25, 2026

(54) A COMPOSITION FOR TREATING PARKINSON'S DISEASE

(71) Applicant: COUNCIL OF SCIENTIFIC & INDUSTRIAL RESEARCH, New Delhi (IN)

(72) Inventors: Deepak KUMAR, Kolkata (IN); Joy CHAKRABORTY, Kolkata (IN); Priyanka YATHAM, Kolkata (IN); Chayan BANERJEE, Kolkata (IN); Shreya Sen SARMA, Kolkata (IN)

(73) Assignee: COUNCIL OF SCIENTIFIC & INDUSTRIAL RESEARCH, New Delhi (IN)

(21) Appl. No.: 19/129,249

(22) PCT Filed: May 14, 2024
(86) PCT No.: PCT/IN2024/050529

§ 371 Date: May 12, 2025

(30) Foreign Application Priority Data
Jun. 2, 2023 (IN) 202311006898

(51) Int. Cl. A61K 31/37; A61K 9/107; A61K 36/23; A61K 47/10; A61K 47/12; A61K 47/14; A61K 47/18; A61K 47/20; A61K 47/22; A61K 47/26; A61K 47/44

(52) U.S. Cl. CPC A61K 31/37; A61K 9/107; A61K 36/23; A61K 47/10; A61K 47/12; A61K 47/14; A61K 47/186; A61K 47/20; A61K 47/22; A61K 47/26; A61K 47/44; A61K 2236/35; A61K 2236/39; A61K 2236/51; A61K 2236/55

(57) ABSTRACT

The present invention provides novel water-soluble compositions comprising sesquiterpene coumarin enriched fractions containing different ratios of sesquiterpene coumarin derived from Ferula species for the treatment of Parkinson's disease via inhibition of MAOenzyme (MAO-A and MAO-B). The invention also provides a method of preparation of ethyl-acetate extract and sesquiterpene coumarin enriched fractions from asafoetida (Hing) that is oleo-gum-resin of Ferula assafoetida using silica gel column chromatography wherein the mobile phase consists of differing compositions of hexane, toluene, chloroform,ethyl-acetate, and methanol. The invention provides the method to prepare the formulation using a multistep process comprising of preparing sesquiterpene coumarin enriched fraction followed by preparing water-soluble formulation. The final composition improves the water solubility, imparts long-term stability, exerts MAO inhibitory activity, and improves pharmaceutical compliance.

### TECHNICAL FIELD OF THE INVENTION

The present invention relates to a method for preparing a water-soluble composition comprising sesquiterpene coumarin enriched fraction containing different ratios of sesquiterpene coumarin derived from Ferula species. The invention further provides a process for the preparation of said water-soluble composition using a process comprising of 1) extraction, fractionation, and preparation of the sesquiterpene coumarin enriched fractions containing different ratios of sesquiterpene coumarin, and 2) preparation of water-soluble compositions. The water-soluble composition increases the solubility, dispersibility, stability, and pharmaceutical compliance of hydrophobic sesquiterpene coumarin. In particular, the invention related to the use of such water-soluble compositions containing sesquiterpene coumarin that inhibits Monoamine oxidase enzyme (MAO; MAO-A and MAO-B) and imparts beneficial effects in the treatment and management of Parkinson's disease.

### BACKGROUND OF THE INVENTION

Parkinson's disease (PD) is the second most common progressive neurodegenerative disorder affecting particularly the elderly population due to the progressive loss of dopaminergic neurons at substantia nigra (SN), which results in the reduction of the dopamine levels in striatal region. This decrease in dopamine level results in tremors, akinesia, and rigidity; the major characteristic features of Parkinson's disease (Dauer & Przedborski, 2003; Lee & Yankee, 2021; Maiti et al., 2017; Mhyre et al., 2012; Rizek et al., 2016). The exponential increase in the population affected by this disorder is creating a huge medical and financial burden on societies. Since the exact cause and site-specificity for Parkinson's disease is not fully known, the current medicines could exert only symptomatic relief to the patients by either a) increasing dopamine levels; b) using dopamine receptor agonist, and c) reduction of dopamine degradation by inhibiting Monoamine oxidase enzyme(Cho et al., 2021; Finberg, 2018; Müller, 2012; Riederer & Laux, 2011). The strategic inhibition of MAO (MAO-A and MAO-B) is implemented in the early stages of Parkinson's disease to keep the dosage of L-dopa under control(Cho et al., 2021; Finberg, 2018; Krishna et al., 2014; Müller, 2012; Nagatsua & Sawadab, 2009; Riederer & Laux, 2011). Several synthetic MAO inhibitory drugs are clinically used but they are not free from adverse effects such as hypertension, disrupted sleep episodes, orthostatic hypotension, arrhythmias, mental status alteration, hallucinations, extrapyramidal symptoms, dyskinesia, and serotonin syndrome. Also, their cessation led to the risk of antidepressant and antiparkinsonian discontinuation syndromes(Koschel et al., 2021; MAO-B Inhibitors (Rasagiline, Selegiline, Safinamide)|Parkinson's UK; Rabinak & Nirenberg, 2010; Solla et al., 2015, 2017). Therefore, to overcome the side effects associated with synthetic drugs, natural products derived from medicinal plants are being developed as potential MAO inhibitors. At the same time, the poor water solubility of natural compounds further hinders their solubility, efficacy, dosage form formulations, and pharmaceutical and patient compliance.

Thus, keeping in view the drawbacks of the hitherto reported prior art, it is realized that there is a dire need to develop a novel natural product-based water-soluble composition for the treatment and management of Parkinson's disease. Therefore, the present invention developedwater-soluble composition comprising sesquiterpene coumarin enriched fractions containing different ratio of sesquiterpene coumarins extracted from Ferulas pp, in particular Ferula assa-foetida.

Sesquiterpene coumarins are the naturally occurring compounds biosynthesized by the plants of the Ferula genus (Family: Umbelliferae or Apiaceae). Among these the most commonly used plant, Ferula assa-foetida, is commonly known as asafoetida and Hing. It finds culinary uses as a characteristic flavoring agent. The exudates or precisely oleo-gum-resin of Ferula assa-foetida are known to contain volatile organosulfide, sesquiterpene, umbelliferone, ferulic acid, and sesquiterpene coumarin (Abd El-Razek et al., 2001; Iranshahy & Iranshahi, 2011; Mohammadhosseini et al., 2015, 2019; Mohammadhosseini & Nekoei, 2014; Yatham et al., 2021). The aqueous methanol extract of Ferula assa-foetida is reported to inhibit acetylcholine esterase enzyme (Ali et al., 2013; Jazayeri et al., 2014); aqueous extract of the Ferula assa-foetida is reported to exert anticonvulsant effects (Bagheri et al., 2014); one of the constituents of Ferula assa-foetida, farnesiferol C, has shown moderate neuroprotective activity against oxygen-glucose deprivation in vitro(Chang et al., 2013); the sesquiterpene coumarin isolated from one of the related species, Ferulasinkiangensis, inhibited the neuroinflammation (Xing et al., 2017); aqueous extract of gum-resin of asafoetida displayed neuroprotective effect and improves neuronal survival rate (Homayouni Moghadam et al., 2013); the extracts of different parts of the Ferula assa-foetida displayed effect in naloxone-induced morphine withdrawal behaviour in mice via Gamma-aminobutyric acid (GABA)(Khanavi et al., 2017); Ferula assa-foetida extracts exerted neuroprotective effects in glutamate-induced neurotoxicity(Tayeboon et al., 2013); oleo-gum-resin of Ferula assa-foetida alleviated the pyridoxine induced peripheral neuropathy in mice (Homayouni Moghadam et al., 2014) and Ferula assa-foetida resin extracted with ethanol at 80° C. inhibited hMAO-B (Zarmouh et al., 2016).

### OBJECTIVES OF THE INVENTION

An objective of the present invention is to develop pharmaceutical compositions comprising differing ratios of sesquiterpene coumarin extracted from Ferula species, in particular Ferula assa-foetida.

Another objective of the present invention is to develop sesquiterpene coumarin enriched compositions that impart superior MAO (MAO-A and MAO-B) inhibitory activities.

Yet another objective of the present invention is to develop pharmaceutical compositions that offer the synergistic effects of sesquiterpene coumarin enriched fractions comprising acyclic, mono-cyclic, and bi-cyclic skeleton-bearing sesquiterpene coumarin wherein the sesquiterpene are naturally attached to a coumarin via an ether linkage.

One more objective is to develop sesquiterpene coumarin enriched fractions which are further formulated into a water-soluble composition with increased water solubility and dispersibility of sesquiterpene coumarin enriched fractions, imparts long-term stability, improves pharmaceutical parameters.

### SUMMARY OF THE INVENTION

Accordingly, the present invention provides a water-soluble composition for the treatment of Parkinson's disease through inhibition of MAO (MAO-A and MAO-B) containing sesquiterpene coumarin having a general chemical structure as—

Sesquiterpene coumarin enriched fraction in the above-depicted water-soluble compositions contains the differing ratio of sesquiterpene coumarins are selected from the following—Ferukrin (1), assafoetidanol A (2), ligupersin A (3), kamolonol (4), 10′-acetoxy-11′-hydroxy umbelliprenin (5), epi-conferdione (6), karatavicinol (7), kellerin (8), ferocaulidin (9), farnesiferol A (10), galbanic acid (11), szowitsiacoumarin B (12), farnesiferol B (13), gummosin (14), farnesiferol C (15), szowitsiacoumarin A (16), methyl galbanate (17).

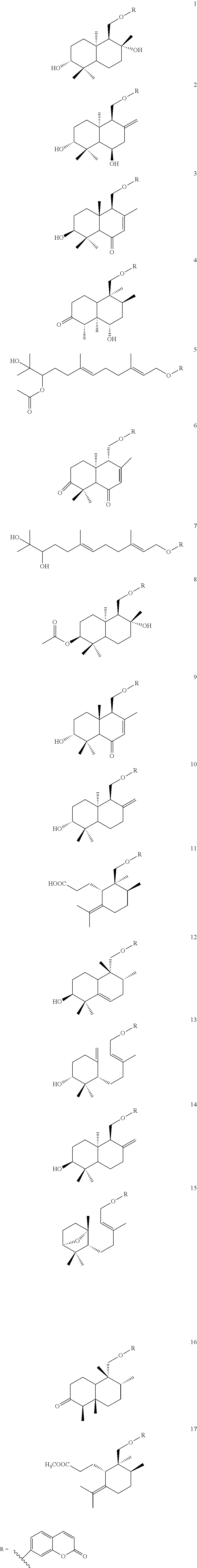

The water-soluble composition inhibits MAO enzymes (MAO-A and MAO-B) and enhances the solubility, dispersibility, stability, and pharmaceutical parameters of sesquiterpene coumarin enriched fractions.

This process essentially differs from the known prior art in the following steps:

### Step 1: Preparing Sesquiterpene Coumarin Enriched Fractions

A method for preparing sesquiterpene coumarin enriched fractions comprising of the following steps (a) Ferulaoleo-gum-resin (asafoetida; Hing) is extracted multiple times with organic solvents of polarity index 2-6 preferably ethyl-acetate (polarity index 4.4), the extracts are combined and concentrated to an ethyl-acetate free residue; (b) ethyl-acetate free residue obtained in step (a) is separated on normal phase silica gel, by elution with a hexane-chloroform (100:0-0:100 v/v) and chloroform-methanol (100:0-70:30 v/v) gradient to obtain sesquiterpene coumarin enriched fractions containing different ratios of sesquiterpene coumarin with umbelliferone and ferulic acid; (c) further separation of fractions obtained in step (b) by octadecylsilane-bonded reverse phase (C18) HPLC eluted with an acetonitrile-water gradient to give pure sesquiterpene coumarin; (d) in step (b) the sesquiterpene coumarin enriched fractions comprises ferukrin (0.05-42.31%), assafoetidanol A (0.013-1.17%), ligupersin A (0.31-36.29%), kamolonol (1.50-46.67%), 10′-acetoxy-11′-hydroxy umbelliprenin (1.52-2.86%), epi-conferdione (0.24-12.24%), karatavicinol (0.07-6.13%), kellerin (1.43-17.63%), ferocaulidin (0.19-2.23%), farnesiferol A (0.13-14.31%), galbanic acid (0.05-52.56%), szowitsiacoumarin B (5.18-16.18%), farnesiferol B (0.81-10.08%), gummosin (2.58-4.03%), farnesiferol C (0.01-40.98%), szowitsiacoumarin A (0.28-7.01%), and methyl galbanate (0.70-10.77%).

### Step 2: Preparing Water-Soluble Compositions

The sesquiterpene coumarin enriched fractions containing different ratios of sesquiterpene coumarin are dissolved in a suitable fatty acid or fatty ester containing oil phase selected from but not restricted to caprylic acid, isopropyl myristate, olive oil, coconut oil, sunflower oil, and ethyl oleate either alone or in combinations in a ratio of 1:1-1:10 w/w with high or low external energy input for 5 min to 24 hours at 10° C. to 40° C. to obtain a clear transparent solution. The solution is further treated with non-ionic, cationic or anionic emulsifiers selected from but not restricted to Tween 20, Tween 60, Tween 80, Span 80, Span 20, sodium lauryl sulfate, Brij35, dodecyl trimethyl ammonium chloride, Trigol, and cetyltrimethylammonium bromidewith or without co-surfactants in the ratio of 1:1.1-1:12.5 w/w with high or low energy agitation for 1 min to 24 hours at 10° C. to 30° C. The resultant mixture is further dispersed in water in the ratio of 1:0.5-1:20% w/w with high or lowenergy agitation for 1 min to 24 hours to obtain a water-soluble composition.

### DETAILED DESCRIPTION OF THE INVENTION

The present invention provides novel water-soluble compositions containing sesquiterpene coumarin enriched fractions for the management or treatment of Parkinson's disease through inhibition of MAO enzyme (MAO-A and MAO-B). More specifically this invention is further directed to the method of preparation of ethyl-acetate extract and sesquiterpene coumarin enriched fractions from the asafoetida (Hing; oleo-gum-resin of Ferula assa-foetida) using silica gel column chromatography wherein the mobile phase consists of differing compositions of hexane, chloroform, and methanol (polarity 2-6). The specific ratios of the sesquiterpene coumarinsin the fractions are determined by HPLC analysis at 325 nm.

Particularly, the present invention provides a method for the preparation of water-soluble compositions comprising sesquiterpene coumarin enriched fractions derived from Ferula species. The invention further provides a process for the preparation of said water-soluble composition using a process comprising of—
1) extraction, fractionation, and preparation of the sesquiterpene coumarin enriched fractions—The asafoetida is extracted multiple times with any known method using organic solvents having polarity index 2-6, in particular ethyl-acetate (polarity index 4.4) followed by evaporation of the solvent to obtain a solvent-free residue. The solvent-free residue is further fractionated by normal phase silica gel column chromatography using different combinations of solvents having a polarity index of 0-6 in particular hexane, chloroform, and methanol. The fractions are concentrated under reduced pressure to obtain solvent-free residues and analyzed by HPLC at 325 nm.
2) The sesquiterpene coumarin enriched fractions containing sesquiterpene coumarin compositions comprising ferukrin (0.05-42.31%), assafoetidanol A (0.013-1.17%), ligupersin A (0.31-36.29%), kamolonol (1.50-46.67%), 10′-acetoxy-11′-hydroxy umbelliprenin (1.52-2.86%), epi-conferdione (0.24-12.24%), karatavicinol (0.07-6.13%), kellerin (1.43-17.63%), ferocaulidin (0.19-2.23%), farnesiferol A (0.13-14.31%), galbanic acid (0.05-52.56%), szowitsiacoumarin B (5.18-16.18%), farnesiferol B (0.81-10.08%), gummosin (2.58-4.03%), farnesiferol C (0.01-40.98%), szowitsiacoumarin A (0.28-7.01%), and methyl galbanate (0.70-10.77%) are treated with a suitable oil phase in the ratio of 1:1-1:10% w/w with high or low energy agitation for 1 min to 24 hours at 10° C. to 30° C. to obtain clear and transparent solution. The solution is further treated with emulsifiers and co-surfactants in the ratio of 1:1-1:12.5 w/w with high or low energy agitation for 1 min to 24 hours at 10° C. to 30° C. The resultant mixture is further dispersed in water in the ratio of 1:0.5-1:20% w/w with high or low energy agitation for 1 min to 24 hours to obtain a water-soluble composition.

The water-soluble compositions thus obtained increase the solubility, dispersibility, stability, and pharmaceutical compliance of hydrophobic sesquiterpene coumarin enriched fractions. In particular, the invention related to the use of such water-soluble compositions containing sesquiterpene coumarin enriched fractions that inhibits MAO enzyme (MAO-A and MAO-B) and imparts beneficial effects for the treatment and management of Parkinson's disease. The said water-soluble composition can be formulated into liquid, solid, gel, and semi-solid dosage forms.

### The Water Soluble Composition Exhibits the Following Characteristics:

The water soluble composition contains differing ratios of sesquiterpene coumarins comprising of bicyclic, monocyclic, and acyclic sesquiterpene skeleton bearing sesquiterpene coumarin enriched fractions dissolved in a suitable oil phase, co-surfactants, emulsifiers, and water.
The composition contains the sesquiterpene coumarins having a general molecular formula C₂₄₋₂₆H₂₆₋₃₆O₃₋₆ and molecular weight in the range of 362-444.
The sesquiterpene coumarins are selected from but not restricted toferukrin (0.05-42.31%), assafoetidanol A (0.013-1.17%), ligupersin A (0.31-36.29%), kamolonol (1.50-46.67%), 10′-acetoxy-11′-hydroxy umbelliprenin (1.52-2.86%), epi-conferdione (0.24-12.24%), karatavicinol (0.07-6.13%), kellerin (1.43-17.63%), ferocaulidin (0.19-2.23%), farnesiferol A (0.13-14.31%), galbanic acid (0.05-52.56%), szowitsiacoumarin B (5.18-16.18%), farnesiferol B (0.81-10.08%), gummosin (2.58-4.03%), farnesiferol C (0.01-40.98%), szowitsiacoumarin A (0.28-7.01%), and methyl galbanate (0.70-10.77%).
Fatty acid or fatty ester containing oil phase is selected from caprylic acid, isopropyl myristate, olive oil, coconut oil, and ethyl oleate either alone or in combinations.
Non-ionic, cationic and anionic emulsifiers are selected from Tween 20, Tween 60, Tween 80, Span 80, Span 20, sodium lauryl sulfate, Brij35, dodecyl trimethyl ammonium chloride, Trigol, and cetyltrimethylammonium bromide
Co-surfactants are selected from propylene glycol and PEG400.
The water soluble compositioncontaining sesquiterpene coumarin enriched fractions inhibits MAO enzyme (MAO-A and MAO-B).
It is miscible in polar solvents such as water but immiscible in nonpolar solvents.
It exhibits stability for at least 1 month at a pH in the range of 2.0 to 10.0.
It can be stored at a temperature in the range of 10 to 40° C. for at least 3 months.
It can be further formulated in different dosage forms such as solid, semi-solid, gel, and liquid.
It can be analyzed by HPLC, NMR, FT-IR, DLS, and TEM.

Thus, the water-soluble composition comprising sesquiterpene coumarin enriched fractions can achieve potential applications in pharmaceutical, nutraceutical, phytochemical, and healthcare industriesfor the management and treatment of Parkinson's disease through inhibition of MAO enzyme (MAO-A and MAO-B).

### The Inventive Steps Involved in the Present Invention are:

This process essentially differs from the known prior art in the following steps:

### Step 1: Preparing Sesquiterpene Coumarin Enriched Fractions

A method for preparing sesquiterpene coumarin enriched fractions comprising of the following steps (a) Ferula oleo-gum-resin (asafoetida; Hing) is extracted multiple times with organic solvents of polarity index 2-6 preferably ethyl-acetate (polarity index 4.4), the extracts are combined and concentrated to an ethyl-acetate free residue; (b) ethyl-acetate free residue obtained in step (a) is separated on normal phase silica gel, by elution with a hexane-chloroform (100:0-0:100 v/v) and chloroform-methanol (100:0-70:30 v/v) gradient to obtain sesquiterpene coumarin enriched fractions containing different ratios of sesquiterpene coumarin with umbelliferone and ferulic acid; (c) further separation of fractions obtained in step (b) by octadecylsilane-bonded reverse phase (C18) HPLC eluted with an acetonitrile-water gradient to give pure sesquiterpene coumarin; (d) in step (b) the sesquiterpene coumarin enriched fractions comprises ferukrin (0.05-42.31%), assafoetidanol A (0.013-1.17%), ligupersin A (0.31-36.29%), kamolonol (1.50-46.67%), 10′-acetoxy-11′-hydroxy umbelliprenin (1.52-2.86%), epi-conferdione (0.24-12.24%), karatavicinol (0.07-6.13%), kellerin (1.43-17.63%), ferocaulidin (0.19-2.23%), farnesiferol A (0.13-14.31%), galbanic acid (0.05-52.56%), szowitsiacoumarin B (5.18-16.18%), farnesiferol B (0.81-10.08%), gummosin (2.58-4.03%), farnesiferol C (0.01-40.98%), szowitsiacoumarin A (0.28-7.01%), and methyl galbanate (0.70-10.77%).

### Step 2: Preparing Water-Soluble Compositions

The sesquiterpene coumarin enriched fractions containing different ratios of sesquiterpene coumarin are dissolved in a suitable fatty acid or fatty ester containing oil phase selected from but not restricted to caprylic acid, isopropyl myristate, olive oil, coconut oil, sunflower oil, and ethyl oleate either alone or in combinations in a ratio of 1:1-1:10 w/w with high or low external energy input for 5 min to 24 hours at 10° C. to 40° C. to obtain a clear transparent solution. The solution is further treated with non-ionic, cationic or anionic emulsifiers selected from but not restricted to Tween 20, Tween 60, Tween 80, Span 80, Span 20, sodium lauryl sulfate, Brij35, dodecyl trimethyl ammonium chloride, Trigol, and cetyltrimethylammonium bromide with or without co-surfactants in the ratio of 1:1.1-1:12.5 w/w with high or low energy agitation for 1 min to 24 hours at 10° C. to 30° C. The resultant mixture is further dispersed in water in the ratio of 1:0.5-1:20% w/w with high or low energy agitation for 1 min to 24 hours to obtain a water-soluble composition.

### EXAMPLES

The following examples are given by way of illustration only and therefore, should not be construed to limit the scope of the present invention.

### Example 1

In a clean round bottom flask, 1.75 kg ferulaoleo-gum-resin(asafoetida) was added and extracted with 3.5 lit of ethyl-acetate at room temperature (25-30° C.) under continuous stirring (500 rpm) for 2 hours. The ethyl-acetate was filtered and the residue was re-extracted three more times. The pooled ethyl-acetate extract was evaporated under reduced pressure at 45-50° C. to obtain oily reddish residue with a strong sulphurous odor. The oily residue was chromatographed over a bed of silica gel using hexane, chloroform, and chloroform: methanol (8:2% v/v) to obtain three sub-fractions. The sub-fraction eluted with chloroform is further subjected to silica gel column chromatography and eluted with increasing polarity of hexane, hexane: chloroform. The sub-fraction eluted in hexane: chloroform (75:25% v/v) was dried under reduced pressure to obtain a gummy residue enriched with sesquiterpene coumarin.

For the preparation of the water-soluble composition, 0.5 gm sesquiterpene coumarin enriched fraction was treated with 3.35 gm of caprylic acid with the aid of high energy sonication for 30 min at 30° C. to obtain a clear and transparent solution. The resultant solution was treated with 6.7 gm Tween 20 at 30° C. with high energy ultrasonication for 1 hour and further dispersed in 210 gm water with high energy ultasonication for 30 min to obtain a milky translucent water-soluble composition.

The obtained sesquiterpene coumarin enriched fraction and its water-soluble composition were characterized and found to exhibit the following characteristics:
The sesquiterpene coumarin enriched fraction is gummy and reddish-brown.
The sesquiterpene coumarin enriched fraction is miscible in nonpolar to mid-polar solvents but immiscible in water.
The sesquiterpene coumarin enriched fraction containsumbelliferone 31.09%, galbanic acid 19.28%, farnesiferol C 40.98%, szowitsiacoumarin A 2.49%, ferocaulidin 0.64%, methyl galbanate 2.13% and karatavicinol 1.24% area at 325 nm in HPLC.
The sesquiterpene coumarin enriched fraction displayed MAO-A (IC₅₀ 0.06952 mg/ml) and MAO-B (IC₅₀ 0.01319 mg/ml) inhibitory activities.
The sesquiterpene coumarin enriched fraction is unstable at a pH in the range of 2.0 to 10.0 and temperature in the range of 30-80° C.
The water-soluble composition containing sesquiterpene coumarin enriched fraction is whitish.
The water-soluble composition containing sesquiterpene coumarin enriched fraction is miscible with water but immiscible with nonpolar solvents.
The water-soluble composition containing sesquiterpene coumarin enriched fraction is stable at a pH range of 2.0 to 10.0.
The water-soluble composition containing sesquiterpene coumarin enriched fraction inhibits MAO enzyme.
The water-soluble composition containing sesquiterpene coumarin enriched fraction displays characteristic peaks in HPLC and NMR.

### Example 2

In a clean round bottom flask, 1.75 kg ferulaoleo-gum-resin (asafoetida) was added and extracted with 3.5 lit of ethyl-acetate at room temperature (25-30° C.) under continuous stirring (500 rpm) for 2 hours. The ethyl-acetate was filtered and the residue was re-extracted three more times. The pooled ethyl-acetate extract was evaporated under reduced pressure at 45-50° C. to obtain oily reddish residue with a strong sulfurous odor. The oily residue was chromatographed over a bed of silica gel using hexane, chloroform, and chloroform: methanol (8:2% v/v) to obtain three sub-fractions. The fraction eluted with chloroform is further subjected to silica gel column chromatography and eluted with increasing polarity of hexane, hexane: chloroform. The sub-fraction eluted in hexane: chloroform (75:25% v/v) was dried under reduced pressure to obtain a gummy residue enriched with sesquiterpene coumarin.

For the preparation of the water-soluble composition, 1 gm sesquiterpene coumarin enriched subfraction was treated with 1.75 gm caprylic acid with the aid of high energy sonication for 10 min at 40° C. to obtain a clear and transparent solution. The resultant mixture was treated with 34.25 gm Tween 60/PG (1:1 w/w) at 25° C. with low energy stirring for 24 hrs and further dispersed in 110 gm water with high energy ultrasonication for 1 min to obtain a translucent water-soluble composition.

The obtained sesquiterpene coumarin enriched fraction and its water-soluble composition were characterized and found to exhibit the following characteristics:
The sesquiterpene coumarin enriched fraction is gummy semisolid and reddish-brown.
The sesquiterpene coumarin enriched fraction is miscible in nonpolar to mid-polar solvents but immiscible in water.
The sesquiterpene coumarin enriched fraction contains ferulic acid 0.67%, umbelliferone 17.76%, galbanic acid 44.56%, farnesiferol B 0.81%, karatavicinol 0.62%, ferocaulidin 0.19%, farnesiferol C19.14%, szowitsiacoumarin A 1.43% and methyl galbanate 1.97% area at 325 nm in HPLC.
The sesquiterpene coumarin enriched fraction displayed MAO-A (IC₅₀0.0403 mg/ml) inhibitory and MAO-B (IC₅₀0.0040 mg/ml) inhibitory activities.
The sesquiterpene coumarin enriched fraction is unstable at a pH in the range of 2.0 to 10.0 and temperature in the range of 30-80° C.
The water-soluble composition containing sesquiterpene coumarin enriched fraction is light yellow.
The water-soluble composition containing sesquiterpene coumarin enriched fraction is miscible with water but immiscible with nonpolar solvents.
The water-soluble composition containing sesquiterpene coumarin enriched fraction is stable at a pH range of 2.0 to 10.0.
The water-soluble composition containing sesquiterpene coumarin enriched fraction inhibits MAO enzyme.
The water-soluble composition containing sesquiterpene coumarin enriched fraction displays characteristic peaks in HPLC and NMR.

### Example 3

In a clean round bottom flask, 1.75 kg ferulaoleo-gum-resin was added and extracted with 3.5 lit of ethyl-acetate at room temperature (25-30° C.) under continuous stirring (500 rpm) for 2 hours. The ethyl-acetate was filtered and the residue was re-extracted three more times. The pooled ethyl-acetate extract was evaporated under reduced pressure at 45-50° C. to obtain oily reddish residue with a strong sulfurous odor. The oily residue was chromatographed over a bed of silica gel using hexane, chloroform, and chloroform: methanol (8:2% v/v) to obtain three sub-fractions. The fraction eluted with chloroform is further subjected to silica gel column chromatography and eluted with increasing polarity of hexane, hexane: chloroform. The sub-fraction eluted in hexane: chloroform (75:25% v/v) was dried under reduced pressure to obtain a gummy residue enriched with sesquiterpene coumarin.

For the preparation of the water-soluble composition, 0.5 gm sesquiterpene coumarin enriched subfraction was treated with 2.5 gm caprylic acid and olive oil (1:1 w/w) with the aid of stirring for 2 hrs at 30° C. to obtain a clear and transparent solution. The resultant mixture was treated with 12.5 gm Tween 20 and sodium lauryl sulfate (8:2 w/w) with the aid of low energy stirring for 180 min at 20° C. and further dispersed in 45 gm water with stirring for 3 hours to obtain a milky white water-soluble composition.

The obtained sesquiterpene coumarin enriched fraction and its water-soluble composition were characterized and found to exhibit the following characteristics:
The sesquiterpene coumarin enriched fraction is gummy solid and brownish.
The sesquiterpene coumarin enriched fraction is miscible in nonpolar to mid-polar solvents but immiscible in water.
The sesquiterpene coumarin enriched fraction contains ferulic acid 0.79%, umbelliferone 19.83%, galbanic acid 52.56%, farnesiferol B 10.08%, farnesiferol C 7.66%, szowitsiacoumarin A 0.72%, and methyl galbanate 1.36% area at 325 nm in HPLC.
The sesquiterpene coumarin enriched fraction displayed MAO-A (IC₅₀0.0643 mg/ml) inhibitory and MAO-B (IC₅₀ 0.0016 mg/ml) inhibitory activities.
The sesquiterpene coumarin enriched fraction is unstable at a pH in the range of 2.0 to 10.0 and temperature in the range of 30-80° C.
The water-soluble composition containing sesquiterpene coumarin enriched fraction is white.
The water-soluble composition containing sesquiterpene coumarin enriched fraction is miscible with water but immiscible with nonpolar solvents.
The water-soluble composition containing sesquiterpene coumarin enriched fraction is stable at a pH range of 2.0 to 10.0.
The water-soluble composition containing sesquiterpene coumarin enriched fraction inhibits MAO enzyme.
The water-soluble composition containing sesquiterpene coumarin enriched fraction displays characteristic peaks in HPLC and NMR.

### Example 4

In a clean round bottom flask, 1.75 kg ferula oleo-gum-resin (asafoetida) was added and extracted with 3.5 lit of ethyl-acetate at room temperature (25-30° C.) under continuous stirring (500 rpm) for 2 hours. The ethyl-acetate was filtered and the residue was re-extracted three more times. The pooled ethyl-acetate extract was evaporated under reduced pressure at 45-50° C. to obtain oily reddish residue with a strong sulphurous odor. The oily residue was chromatographed over a bed of silica gel using hexane, chloroform, and chloroform: methanol (8:2% v/v) to obtain three sub-fractions. The sub-fraction eluted with chloroform is further subjected to silica gel column chromatography and eluted with increasing polarity of hexane, hexane: chloroform. The sub-fraction eluted in hexane: chloroform (75:25% v/v) was dried under reduced pressure to obtain a gummy residue enriched with sesquiterpene coumarin.

For the preparation of the water-soluble composition, 0.5 gm sesquiterpene coumarin enriched fraction was treated with 1.5 gm of olive oil with the aid of low energy stirring for 6 hrs at 20° C. to obtain a clear and transparent solution. The resultant mixture was treated with 12 gm Brij 35, propylene glycol and sodium lauryl sulfate (2:2:1 w/w) with the aid of high energy ultrasonication for 15 min at 15° C. and further dispersed in 24 gm water with low energy stirring for 45 min to obtain a milky white water-soluble composition.

The obtained sesquiterpene coumarin enriched fraction and its water-soluble composition were characterized and found to exhibit the following characteristics:
The sesquiterpene coumarin enriched fraction is oily and reddish-brown.
The sesquiterpene coumarin enriched fraction is miscible in nonpolar to mid-polar solvents but immiscible in water.
The sesquiterpene coumarin enriched fraction contains ferulic acid 0.58%, umbelliferone 30.96%, galbanic acid 43.67%, farnesiferol B5.94%, szowitsiacoumarin A 0.39%, farnesiferol C1.49% and methyl galbanate 2.37% area at 325 nm in HPLC.
The sesquiterpene coumarin enriched fraction displayed MAO-A (IC₅₀ 0.0631 mg/ml) inhibitory and MAO-B (IC₅₀ 0.0024 mg/ml) inhibitory activities.
The sesquiterpene coumarin enriched fraction is unstable at a pH in the range of 2.0 to 10.0 and temperature in the range of 30-80° C.
The water-soluble composition containing sesquiterpene coumarin enriched fraction is white.
The water-soluble composition containing sesquiterpene coumarin enriched fraction is miscible with water but immiscible with nonpolar solvents.
The water-soluble composition containing sesquiterpene coumarin enriched fraction is stable at a pH range of 2.0 to 10.0.
The water-soluble composition containing sesquiterpene coumarin enriched fraction inhibits MAO enzyme.
The water-soluble composition containing sesquiterpene coumarin enriched fraction displays characteristic peaks in HPLC and NMR.

### Example 5

In a clean round bottom flask, 1.75 kg ferula oleo-gum-resin (asafoetida) was added and extracted with 3.5 lit of ethyl-acetate at room temperature (25-30° C.) under continuous stirring (500 rpm) for 2 hours. The ethyl-acetate was filtered and the residue was re-extracted three more times. The pooled ethyl-acetate extract was evaporated under reduced pressure at 45-50° C. to obtain oily reddish residue with a strong sulphurous odor. The oily residue was chromatographed over a bed of silica gel using hexane, chloroform, and chloroform: methanol (8:2% v/v) to obtain three sub-fractions. The sub-fraction eluted with chloroform is further subjected to silica gel column chromatography and eluted with increasing polarity of hexane, hexane: chloroform. The sub-fraction eluted in hexane: chloroform (50:50% v/v) was dried under reduced pressure to obtain a gummy residue enriched with sesquiterpene coumarin.

For the preparation of the water-soluble composition, 2.5 gm sesquiterpene coumarin enriched fraction was treated with 15 gm of olive oil and caprylic acid (0.1:1 w/w) with the aid of low energy stirring for 12 hrs at 25° C. to obtain a clear and transparent solution. The resultant mixture was treated with 35 gm Span 80 and dodecyl trimethyl smmonium chloride (8:2 w/w) with the aid of high energy ultrasonication for 45 min at 30° C. and further dispersed in 525 gm water with loe energy stirring for 24 hours to obtain an opaque milky white water-soluble composition.

The obtained sesquiterpene coumarin enriched fraction and its water-soluble composition were characterized and found to exhibit the following characteristics:
The sesquiterpene coumarin enriched fraction is oily and reddish-brown.
The sesquiterpene coumarin enriched fraction is miscible in nonpolar to mid-polar solvents but immiscible in water.
The sesquiterpene coumarin enriched fraction contains umbelliferone 16.25%, galbanic acid 49.42%, farnesiferol B1.63%, farnesiferol C 5.61%, epi-conferdione 2.28%, ferocaulidin 1.16% farnesiferol A 3.79% and methyl galbanate 10.77% area at 325 nm in HPLC.
The sesquiterpene coumarin enriched fraction displayed MAO-A (IC₅₀ 0.0075 mg/ml) inhibitory and MAO-B (IC₅₀ 0.0047 mg/ml) inhibitory activities.
The sesquiterpene coumarin enriched fraction is unstable at a pH in the range of 2.0 to 10.0 and temperature in the range of 30-80° C.
The water-soluble composition containing sesquiterpene enriched fraction coumarin is whitish.
The water-soluble composition containing sesquiterpene coumarin enriched fraction is miscible with water but immiscible with nonpolar solvents.
The water-soluble composition containing sesquiterpene coumarinenriched fraction is stable at a pH range of 2.0 to 10.0.
The water-soluble composition containing sesquiterpene coumarinenriched fraction inhibits MAO enzyme.
The water-soluble composition containing sesquiterpene coumarin enriched fraction displays characteristic peaks in HPLC and NMR analysis.

### Example 6

In a clean round bottom flask, 1.75 kg ferula oleo-gum-resin (asafoetida) was added and extracted with 3.5 lit of ethyl-acetate at room temperature (25-30° C.) under continuous stirring (500 rpm) for 2 hours. The ethyl-acetate was filtered and the residue was re-extracted three more times. The pooled ethyl-acetate extract was evaporated under reduced pressure at 45-50° C. to obtain oily reddish residue with a strong sulphurous odor. The oily residue was chromatographed over a bed of silica gel using hexane, chloroform, and chloroform: methanol (8:2% v/v) to obtain three sub-fractions. The sub-fraction eluted with chloroform is further subjected to silica gel column chromatography and eluted with increasing polarity of hexane, hexane: chloroform. The sub-fraction eluted in hexane: chloroform (50:50% v/v) was dried under reduced pressure to obtain a gummy residue enriched with sesquiterpene coumarin.

For the preparation of the water-soluble composition, 2.5 gm sesquiterpene coumarin enriched fraction was treated with 2.5 gm isopropylmyristate with the aid of high energy ultrasonication for 8 hrs at 15° C. to obtain a clear and transparent solution. The resultant mixture was treated with 22.5 gm Tween 60 and Trigol (1:1 w/w) with the aid of high energy ultrasonication for 120 min at 30° C. and further dispersed in 16.5 gm water with low energy stirring for 10 hours to obtain a translucent brownish water-soluble composition.

The obtained sesquiterpene coumarin enriched fraction and its water-soluble composition were characterized and found to exhibit the following characteristics:
The sesquiterpene coumarin enriched fraction is oily and reddish-brown.
The sesquiterpene coumarin enriched fraction is miscible in nonpolar to mid-polar solvents but immiscible in water.
The sesquiterpene coumarin enriched fraction contains ferulic acid 1.16%, umbelliferone 7.26%, kamolonol 2.30%, ferocaulidin 1.03%, epi-conferdione 12.24%, kellerin 17.63%, karatavicinol 0.42%, farnesiferol A 14.31%, galbanic acid 28.46%, fernesiferol C 1.10%, methyl galbanate 4.96% area at 325 nm in HPLC.
The sesquiterpene coumarin enriched fraction displayed MAO-A (IC₅₀ 0.0101 mg/ml) inhibitory and MAO-B (IC₅₀ 0.0012 mg/ml) inhibitory activities.
The sesquiterpene coumarin enriched fraction is unstable at a pH in the range of 2.0 to 10.0 and temperature in the range of 30-80° C.
The water-soluble composition containing sesquiterpene coumarin enriched fraction is yellowish white.
The water-soluble composition containing sesquiterpene coumarin enriched fraction is miscible with water but immiscible with nonpolar solvents.
The water-soluble composition containing sesquiterpene coumarinenriched fraction is stable at a pH range of 2.0 to 10.0.
The water-soluble composition containing sesquiterpene coumarin enriched fraction inhibits MAO enzyme.
The water-soluble composition containing sesquiterpene coumarin enriched fraction displays characteristic peaks in HPLC and NMR.

### Example 7

In a clean round bottom flask, 1.75 kg ferula oleo-gum-resin (asafoetida) was added and extracted with 3.5 lit of ethyl-acetate at room temperature (25-30° C.) under continuous stirring (500 rpm) for 2 hours. The ethyl-acetate was filtered and the residue was re-extracted three more times. The pooled ethyl-acetate extract was evaporated under reduced pressure at 45-50° C. to obtain oily reddish residue with a strong sulphurous odor. The oily residue was chromatographed over a bed of silica gel using hexane, chloroform, and chloroform: methanol (8:2% v/v) to obtain three sub-fractions. The sub-fraction eluted with chloroform is further subjected to silica gel column chromatography and eluted with increasing polarity of hexane, hexane: chloroform. The sub-fraction eluted in hexane: chloroform (50:50% v/v) was dried under reduced pressure to obtain a gummy residue enriched with sesquiterpene coumarin.

For the preparation of the water-soluble composition, 0.5 gm sesquiterpene coumarin enriched fraction was treated with 5 gm of caprylic acid and isopropyl myristate (1:1 w/w) with the aid of low energy stirring for 20 hrs at 20° C. to obtain a clear and transparent solution. The resultant solution was treated with 44 gm Span 20, PEG400 and sodium lauryl sulfate (2:6:2 w/w) with the aid of high energy ultrasonication for 90 min at 15° C. and further dispersed in 198 gm water with low energy stirring for 10 min to obtain a clear transparent water-soluble composition. The obtained sesquiterpene coumarin enriched fraction and its water-soluble composition were characterized and found to exhibit the following characteristics:
The sesquiterpene coumarin enriched fraction is oily and reddish-brown.
The sesquiterpene coumarin enriched fraction is miscible in nonpolar to mid-polar solvents but immiscible in water.
The sesquiterpene coumarin enriched fraction contains ferulic acid 1.05%, umbelliferone 11.10%, kamolonol 18.42%, epi-conferdione 5.87%, kellerin 12.70%, karatavicinol 2.34%, farnesiferol A 9.68%, szowitsiacoumarin B 16.18%, farnesiferol C 1.0%, szowitsiacoumarin A 0.61%, methyl galbanate 9.08% area at 325 nm in HPLC.
The sesquiterpene coumarin enriched fraction displayed MAO-A (IC₅₀ 0.0095 mg/ml) inhibitory and MAO-B (IC₅₀ 0.0075 mg/ml) inhibitory activities.
The sesquiterpene coumarin enriched fraction is unstable at a pH in the range of 2.0 to 10.0 and temperature in the range of 30-80° C.
The water-soluble composition containing sesquiterpene coumarin enriched fraction is light yellow.
The water-soluble composition containing sesquiterpene coumarin enriched fraction is miscible with water but immiscible with nonpolar solvents.
The water-soluble composition containing sesquiterpene coumarin enriched fraction is stable at a pH range of 2.0 to 10.0.
The water-soluble composition containing sesquiterpene coumarin enriched fraction inhibits MAO enzyme.
The water-soluble composition containing sesquiterpene coumarin enriched fraction displays characteristic peaks in HPLC and NMR.

### Example 8

In a clean round bottom flask, 1.75 kg ferula oleo-gum-resin (asafoetida) was added and extracted with 3.5 lit of ethyl-acetate at room temperature (25-30° C.) under continuous stirring (500 rpm) for 2 hours. The ethyl-acetate was filtered and the residue was re-extracted three more times. The pooled ethyl-acetate extract was evaporated under reduced pressure at 45-50° C. to obtain oily reddish residue with a strong sulphurous odor. The oily residue was chromatographed over a bed of silica gel using hexane, chloroform, and chloroform: methanol (8:2% v/v) to obtain three sub-fractions. The sub-fraction eluted with chloroform is further subjected to silica gel column chromatography and eluted with increasing polarity of hexane, hexane: chloroform. The sub-fraction eluted in hexane: chloroform (50:50% v/v) was dried under reduced pressure to obtain a gummy residue enriched with sesquiterpene coumarin.

For the preparation of the water-soluble composition, 0.5 gm sesquiterpene coumarin enriched fraction was treated with 4.5 gm of isopropyl myristate with the aid of low energy stirring for 24 hrs at 10° C. to obtain a clear and transparent solution. The resultant solution was treated with 50 gm Tween 80 with the aid of high energy ultrasonication for 1 min at 30° C. and further dispersed in 110 gm water with low energy stirring for 30 min to obtain a clear transparent water-soluble composition.

The obtained sesquiterpene coumarin enriched fraction and its water-soluble composition were characterized and found to exhibit the following characteristics:
The sesquiterpene coumarin enriched fraction is oily and reddish-brown.
The sesquiterpene coumarin enriched fraction is miscible in nonpolar to mid-polar solvents but immiscible in water.
The sesquiterpene coumarin enriched fraction contains ferulic acid 0.99%, umbelliferone 13.08%, kamolonol 46.67%, epi-conferdione 0.24%, kellerin 1.43%, karatavicinol 1.92%, acetoxy-umbelliprenin 1.52%, gummosin 4.03%, szowitsiacoumarin B 5.18%, szowitsiacoumarin A 7.01% and methyl galbanate 1.13% area at 325 nm in HPLC.
The sesquiterpene coumarin enriched fraction displayed MAO-A (IC₅₀ 0.0901 mg/ml) inhibitory and MAO-B (IC₅₀ 0.0373 mg/ml) inhibitory activities.
The sesquiterpene coumarin enriched fraction is unstable at a pH in the range of 2.0 to 10.0 and temperature in the range of 30-80° C.
The water-soluble composition containing sesquiterpene coumarin enriched fraction is yellowish.
The water-soluble composition containing sesquiterpene coumarin enriched fraction is miscible with water but immiscible with nonpolar solvents.
The water-soluble composition containing sesquiterpene coumarin enriched fraction is stable at a pH range of 2.0 to 10.0.
The water-soluble composition containing sesquiterpene coumarin enriched fraction inhibits MAO enzyme.
The water-soluble composition containing sesquiterpene coumarin enriched fraction displays characteristic peaks in HPLC and NMR.

### Example 9

In a clean round bottom flask, 1.75 kg ferula oleo-gum-resin (asafoetida) was added and extracted with 3.5 lit of ethyl-acetate at room temperature (25-30° C.) under continuous stirring (500 rpm) for 2 hours. The ethyl-acetate was filtered and the residue was re-extracted three more times. The pooled ethyl-acetate extract was evaporated under reduced pressure at 45-50° C. to obtain oily reddish residue with a strong sulphurous odor. The oily residue was chromatographed over a bed of silica gel using hexane, chloroform, and chloroform: methanol (8:2% v/v) to obtain three sub-fractions. The sub-fraction eluted with chloroform is further subjected to silica gel column chromatography and eluted with increasing polarity of hexane, hexane: chloroform. The sub-fraction eluted in hexane: chloroform (50:50% v/v) was dried under reduced pressure to obtain a gummy residue enriched with sesquiterpene coumarin.

For the preparation of the water-soluble composition, 0.5 gm sesquiterpene coumarin enriched fraction was treated with 4.3 gm of coconut oil and caprylic acid (1:1 w/w) with the aid of low energy stirring for 3.5 hrs at 35° C. to obtain a clear and transparent solution. The resultant solution was treated with 15.75 gm Tween 80, propylene glycol and dodecyl trimethyl ammonium chloride (6:2:2 w/w) with the aid of high energy ultrasonication for 30 min at 20° C. and further dispersed in 98 gm water with low energy stirring for 12 hours to obtain a clear transparent water-soluble composition.

The obtained sesquiterpene coumarin enriched fraction and its water-soluble composition were characterized and found to exhibit the following characteristics:
The sesquiterpene coumarin enriched fraction is oily and reddish-brown.
The sesquiterpene coumarin enriched fraction is miscible in nonpolar to mid-polar solvents but immiscible in water.
The sesquiterpene coumarin enriched fraction contains ferulic acid 1.74%, umbelliferone 19.55%, acetoxy-umbelliprenin 2.86%, kamolonol 43.28%, karatavicinol 6.13%, gummosin 2.58%, szowitsiacoumarin B 5.94%, szowitsiacoumarin A 4.72% and farnesiferol A 0.13% area at 325 nm in HPLC.
The sesquiterpene coumarin enriched fraction displayed MAO-A (IC₅₀ 0.0300 mg/ml) inhibitory and MAO-B (IC₅₀ 0.0038 mg/ml) inhibitory activities.
The sesquiterpene coumarin enriched fraction is unstable at a pH in the range of 2.0 to 10.0 and temperature in the range of 30-80° C.
The water-soluble composition containing sesquiterpene coumarinenriched fraction is light brown.
The water-soluble composition containing sesquiterpene coumarin enriched fraction is miscible with water but immiscible with nonpolar solvents.
The water-soluble composition containing sesquiterpene coumarin enriched fraction is stable at a pH range of 2.0 to 10.0.
The water-soluble composition containing sesquiterpene coumarin enriched fraction inhibits MAO enzyme.
The water-soluble composition containing sesquiterpene coumarin enriched fraction displays characteristic peaks in HPLC and NMR.

### Example 10

In a clean round bottom flask, 1.75 kg ferula oleo-gum-resin (asafoetida) was added and extracted with 3.5 lit of ethyl-acetate at room temperature (25-30° C.) under continuous stirring (500 rpm) for 2 hours. The ethyl-acetate was filtered and the residue was re-extracted three more times. The pooled ethyl-acetate extract was evaporated under reduced pressure at 45-50° C. to obtain oily reddish residue with a strong sulphurous odor. The oily residue was chromatographed over a bed of silica gel using hexane, chloroform, and chloroform: methanol (8:2% v/v) to obtain three sub-fractions. The sub-fraction eluted with chloroform is further subjected to silica gel column chromatography and eluted with increasing polarity of hexane, hexane: chloroform. The sub-fraction eluted in chloroform was dried under reduced pressure to obtain a gummy residue enriched with sesquiterpene coumarin.

For the preparation of the water-soluble composition, 0.5 gm sesquiterpene coumarin enriched fraction was treated with 2 gm of coconut oil with the aid of high energy ultrasonication for 4 hrs at 30° C. to obtain a clear and transparent solution. The resultant solution was treated with 10 gm Tween 60 and PEG400 (1:1 w/w) with the aid of high energy ultrasonication for 30 min at 20° C. and further dispersed in 37.5 gm water with low energy stirring for 5 hours to obtain a clear transparent water-soluble composition.

The obtained sesquiterpene coumarin enriched fraction and its water-soluble composition were characterized and found to exhibit the following characteristics:
The sesquiterpene coumarin enriched fraction is oily and reddish-brown.
The sesquiterpene coumarin enriched fraction is miscible in nonpolar to mid-polar solvents but immiscible in water.
The sesquiterpene coumarin enriched fraction contains ferulic acid 39.10%, umbelliferone 8.97%, ferukrin 6.89%, kamolonol 1.50%, ligupersin A 36.29%, karatavicinol 0.75%, farnesiferol A 1.37%, farnesiferol C 0.23%, szowitsiacoumarin A 0.48% and methyl galbanate 0.70% area at 325 nm in HPLC.
The sesquiterpene coumarin enriched fraction displayed MAO-A (IC₅₀ 0.0699 mg/ml) inhibitory and MAO-B (IC₅₀ 0.0068 mg/ml) inhibitory activities.
The sesquiterpene coumarin enriched fraction is unstable at a pH in the range of 2.0 to 10.0 and temperature in the range of 30-80° C.
The water-soluble composition containing sesquiterpene coumarin enriched fraction is light brown.
The water-soluble composition containing sesquiterpene coumarin enriched fraction is miscible with water but immiscible with nonpolar solvents.
The water-soluble composition containing sesquiterpene coumarin enriched fraction is stable at a pH range of 2.0 to 10.0.
The water-soluble composition containing sesquiterpene coumarin enriched fraction inhibits MAO enzyme.
The water-soluble composition containing sesquiterpene coumarin enriched fraction displays characteristic peaks in HPLC and NMR.

### Example 11

In a clean round bottom flask, 1.75 kg ferula oleo-gum-resin (asafoetida) was added and extracted with 3.5 lit of ethyl-acetate at room temperature (25-30° C.) under continuous stirring (500 rpm) for 2 hours. The ethyl-acetate was filtered and the residue was re-extracted three more times. The pooled ethyl-acetate extract was evaporated under reduced pressure at 45-50° C. to obtain oily reddish residue with a strong sulphurous odor. The oily residue was chromatographed over a bed of silica gel using hexane, chloroform, and chloroform: methanol (8:2% v/v) to obtain three sub-fractions. The sub-fraction eluted with chloroform is further subjected to silica gel column chromatography and eluted with increasing polarity of hexane, hexane: chloroform. The sub-fraction eluted in chloroform was dried under reduced pressure to obtain a gummy residue enriched with sesquiterpene coumarin.

For the preparation of the water-soluble composition, 0.5 gm sesquiterpene coumarin enriched fraction was treated with 4 gm of ethyl oleate with the aid of low energy stirring for 15 hrs at 40° C. to obtain a clear and transparent solution. The resultant solution was treated with 27 gm Brij35 and PEG400 (1:1 w/w) with the aid of high energy ultrasonication for 60 min at 10° C. and further dispersed in 126 gm water with low energy stirring for 2 hours to obtain a translucent milky white water-soluble composition.

The obtained sesquiterpene coumarin enriched fraction and its water-soluble composition were characterized and found to exhibit the following characteristics:
The sesquiterpene coumarin enriched fraction is sticky solid and reddish-brown.
The sesquiterpene coumarin enriched fraction is miscible in nonpolar to mid-polar solvents but immiscible in water.
The sesquiterpene coumarin enriched fraction contains ferulic acid 85.20%, umbelliferone 2.09%, ferukrin 7.37%, assafoetidanol A 1.17%, ligupersin A 1.48%, karatavicinol 0.52%, farnesiferol A 0.27% and galbanic acid 0.17% area at 325 nm in HPLC.
The sesquiterpene coumarin enriched fraction displayed MAO-A (IC₅₀ 0.0568 mg/ml) inhibitory and MAO-B (IC₅₀ 0.0141 mg/ml) inhibitory activities.
The sesquiterpene coumarin enriched fraction is unstable at a pH in the range of 2.0 to 10.0 and temperature in the range of 30-80° C.
The water-soluble composition containing sesquiterpene coumarin enriched fraction is white.
The water-soluble composition containing sesquiterpene coumarin enriched fraction is miscible with water but immiscible with nonpolar solvents.
The water-soluble composition containing sesquiterpene coumarin enriched fraction is stable at a pH range of 2.0 to 10.0.
The water-soluble composition containing sesquiterpene coumarin enriched fraction inhibits MAO enzyme.
The water-soluble composition containing sesquiterpene coumarin enriched fraction displays characteristic peaks in HPLC and NMR.

### Example 12

In a clean round bottom flask, 1.75 kg ferula oleo-gum-resin (asafoetida) was added and extracted with 3.5 lit of ethyl-acetate at room temperature (25-30° C.) under continuous stirring (500 rpm) for 2 hours. The ethyl-acetate was filtered and the residue was re-extracted three more times. The pooled ethyl-acetate extract was evaporated under reduced pressure at 45-50° C. to obtain oily reddish residue with a strong sulphurous odor. The oily residue was chromatographed over a bed of silica gel using hexane, chloroform, and chloroform: methanol (8:2% v/v) to obtain three sub-fractions. The sub-fraction eluted with chloroform: methanol (8:2% v/v) is further subjected to silica gel column chromatography and eluted with increasing polarity of hexane, hexane: chloroform. The sub-fraction eluted in chloroform was dried under reduced pressure to obtain a gummy residue enriched with sesquiterpene coumarin.

For the preparation of the water-soluble composition, 0.5 gm sesquiterpene coumarin enriched fraction was treated with 3.5 gm caprylic acid with the aid of low energy stirring for 8 hrs at 10° C. to obtain a clear and transparent solution. The resultant solution was treated with 4 gm sodium lauryl sulfateat 25° C. with low energy stirring for 1 hour and further dispersed in 27.5 gm water with high energy ultrasonication for 30 min to obtain a translucent white water-soluble composition.

The obtained sesquiterpene coumarin enriched fraction and its water-soluble composition were characterized and found to exhibit the following characteristics:
The sesquiterpene coumarin enriched fraction is oily and reddish-brown.
The sesquiterpene coumarin enriched fraction is miscible in nonpolar to mid-polar solvents but immiscible in water.
The sesquiterpene coumarin enriched fraction contains ferulic acid 98.61%, ferukrin 0.05%, asafoetidanol A 0.13%, ligupersin A 0.31%, karatavicinol 0.07%, farnesiferol C 0.01%, and galbanic acid 0.05% area at 325 nm in HPLC.
The sesquiterpene coumarin enriched fraction displayed MAO-A (IC₅₀ 0.0526 mg/ml) inhibitory and MAO-B (IC₅₀ 0.0198 mg/ml) inhibitory activities.
The sesquiterpene coumarin enriched fraction is unstable at a pH in the range of 2.0 to 10.0 and temperature in the range of 30-80° C.
The water-soluble composition containing sesquiterpene coumarin enriched fraction is yellowish brown.
The water-soluble composition containing sesquiterpene coumarin enriched fraction is miscible with water but immiscible with nonpolar solvents.
The water-soluble composition containing sesquiterpene coumarin enriched fraction is stable at a pH range of 2.0 to 10.0.
The water-soluble composition containing sesquiterpene coumarin enriched fraction inhibits MAO enzyme.
The water-soluble composition containing sesquiterpene coumarin enriched fraction displays characteristic peaks in HPLC and NMR.

### Example 13

In a clean round bottom flask, 1.75 kg ferula oleo-gum-resin (asafoetida) was added and extracted with 3.5 lit of ethyl-acetate at room temperature (25-30° C.) under continuous stirring (500 rpm) for 2 hours. The ethyl-acetate was filtered and the residue was re-extracted three more times. The pooled ethyl-acetate extract was evaporated under reduced pressure at 45-50° C. to obtain oily reddish residue with a strong sulphurous odor. The oily residue was chromatographed over a bed of silica gel using hexane, chloroform, and chloroform: methanol (8:2% v/v) to obtain three sub-fractions. The sub-fraction eluted with chloroform is further subjected to silica gel column chromatography and eluted with increasing polarity of hexane, hexane: chloroform. The sub-fraction eluted in chloroform was dried under reduced pressure to obtain a gummy residue enriched with sesquiterpene coumarin.

For the preparation of the water-soluble composition, 0.5 gm sesquiterpene coumarin enriched fraction was treated with 2 gm caprylic acid and ethyl oleate (1:1 w/w) with the aid of high energy sonication for 5 min at 35° C. to obtain a clear and transparent solution. The resultant solution was treated with 5 gm dodecyl trimethyl ammonium chlorideat 30° C. with low energy stirring for 20 hour and further dispersed in 60 gm with high energy ultrasonication for 60 min water to obtain a clear and transparent water-soluble composition.

The obtained sesquiterpene coumarin enriched fraction and its water-soluble composition were characterized and found to exhibit the following characteristics:
The sesquiterpene coumarin enriched fraction is oily and reddish-brown.
The sesquiterpene coumarin enriched fraction is miscible in nonpolar to mid-polar solvents but immiscible in water.
The sesquiterpene coumarin enriched fraction contains ferulic acid 45.88%, umbelliferone 0.76%, ligupersin A 3.52%, karatavicinol 0.59%, ferocaulidin 1.54%, ferukrin 42.31%, and galbanic acid 3.14% area at 325 nm in HPLC.
The sesquiterpene coumarin enriched fraction displayed MAO-A (IC₅₀ 0.1014 mg/ml) inhibitory and MAO-B (IC₅₀ 0.0135 mg/ml) inhibitory activities.
The sesquiterpene coumarin enriched fraction is unstable at a pH in the range of 2.0 to 10.0 and temperature in the range of 30-80° C.
The water-soluble composition containing sesquiterpene coumarin enriched fraction is light brown.
The water-soluble composition containing sesquiterpene coumarin is miscible with water but immiscible with nonpolar solvents.
The water-soluble composition containing sesquiterpene coumarin enriched fraction is stable at a pH range of 2.0 to 10.0.
The water-soluble composition containing sesquiterpene coumarin enriched fraction inhibits MAO enzyme.
The water-soluble composition containing sesquiterpene coumarin enriched fraction displays characteristic peaks in HPLC and NMR.

Thus the developed composition in the present invention contains sesquiterpene coumarin derived from the Ferula species in particular Ferula assafoetida have MAO (MAO-A and MAO-B) inhibitory activities. The sesquiterpene coumarin contains acyclic, mono-cyclic, and bi-cyclic skeleton-bearing sesquiterpene that are naturally attached to a coumarin via an ether linkage. The sesquiterpene coumarins are nonpolar compounds that have poor water solubility which may hinder their absorption and pharmaceutical compliance. Also, the sesquiterpene coumarin contains an ether linkage which is prone to hydrolysis in the acidic condition of the stomach. Thus the present invention aims to formulate the said sesquiterpene coumarin enriched fractions in a water-soluble composition to impart improved solubility, dispersibility, stability, and pharmaceutical compliance.

### ABBREVIATIONS

MAO: Monoamine oxidase
HPLC: High-performance liquid chromatography
nm: Nano-meter
FT-IR: Fourier-transform infrared spectroscopy
NMR: Nuclear Magnetic Resonance
IC₅₀: Half-maximal inhibitory concentration
gm: Gram
w/w: weight/weight

### Advantages of the Invention

The developed water-soluble compositions have several advantages, which are listed below in brief:
The water-soluble compositions containing sesquiterpene coumarin enriched fractions do not require any harmful chemicals.
The water-soluble compositions containing sesquiterpene coumarin are cost-effective.
The preparation method is very simple and economically viable.
The water-soluble compositions increase the solubility, dispersibility, stability, and pharmaceutical compliance of hydrophobic sesquiterpene coumarin enriched fractions.
The water-soluble compositions containing sesquiterpene coumarin can be formulated into liquid, solid, gel, and semi-solid dosage forms.
The sesquiterpene coumarin enriched fractions and water soluble compositions containing sesquiterpene coumarin enriched fractions exhibit MAO enzyme (MAO-A and MAO-B) inhibitory activities.
The water-soluble compositions containing sesquiterpene coumarin exhibit stability for at least 1 months at a pH in the range of 2.0 to 10.0.
The water-soluble composition containing sesquiterpene coumarin can be stored at a temperature in the range of 10 to 40° C. for at least 3 months.

### REFERENCES

1. Abd El-Razek, M. H., Ohta, S., Ahmed, A. A., & Hirata, T. (2001). Sesquiterpene coumarins from the roots of Ferula assa-foetida. Phytochemistry, 58(8), 1289-1295. https://doi.org/10.1016/S0031-9422(01)00324-7
2. Ali, S. K., Hamed, A. R., Soltan, M. M., Hegazy, U. M., Elgorashi, E. E., El-Garf, I. A., & Hussein, A. A. (2013). In-vitro evaluation of selected Egyptian traditional herbal medicines for treatment of alzheimer disease. BMC Complementary and Alternative Medicine, 13(1), 1-10. https://doi.org/10.1186/1472-6882-13-121/FIGURES/1
3. Bagheri, S. M., Rezvani, M. E., Vahidi, A. R., & Esmaili, M. (2014). Anticonvulsant effect of ferula assa-foetida oleo gum resin on chemical and amygdala-kindled rats. North American Journal of Medical Sciences, 6(8), 408. https://doi.org/10.4103/1947-2714.139296
4. Chang, H. J., Kim, Y. S., Ryu, S. Y., & Chun, H. S. (2013). Screening of various sources of phytochemicals for neuroprotective activity against oxygen-glucose deprivation in vitro. Journal of the Korean Society for Applied Biological Chemistry 2013 56:4, 56(4), 451-455. https://doi.org/10.1007/S13765-013-3108-7
5. Cho, H. U., Kim, S., Sim, J., Yang, S., An, H., Nam, M. H., Jang, D. P., & Lee, C. J. (2021). Redefining differential roles of MAO-A in dopamine degradation and MAO-B in tonic GABA synthesis. Experimental & Molecular Medicine 2021 53:7, 53(7), 1148-1158. https://doi.org/10.1038/s12276-021-00646-3
6. Dauer, W., & Przedborski, S. (2003). Parkinson's Disease: Mechanisms and Models. Neuron, 39(6), 889-909. https://doi.org/10.1016/SO896-6273(03)00568-3
7. Finberg, J. P. M. (2018). Inhibitors of MAO-B and COMT: their effects on brain dopamine levels and uses in Parkinson's disease. Journal of Neural Transmission 2018 126:4, 126(4), 433-448. https://doi.org/10.1007/S00702-018-1952-7
8. Homayouni Moghadam, F., Dehghan, M., Zarepur, E., Dehlavi, R., Ghaseminia, F., Ehsani, S., Mohammadzadeh, G., & Barzegar, K. (2014). Oleo gum resin of Ferula assa-foetida L. ameliorates peripheral neuropathy in mice. Journal of Ethnopharmacology, 154(1), 183-189. https://doi.org/10.1016/j.jep.2014.03.069
9. Homayouni Moghadam, F., Vakili Zarch, B., & Shafiei, M. (2013). Double edged effect of gum-resin of ferula assa-foetida on lifespan of neurons. Iranian Journal of Basic Medical Sciences, 16(4), 668-671. https://doi.org/10.22038/IJBMS.2013.728
10. Iranshahy, M., & Iranshahi, M. (2011). Traditional uses, phytochemistry and pharmacology of asafoetida (Ferula assa-foetida oleo-gum-resin)—A review. In Journal of Ethnopharmacology (Vol. 134, Issue 1, pp. 1-10). Elsevier. https://doi.org/10.1016/j.jep.2010.11.067
11. Jazayeri, S. B., Amanlou, A., Ghanadian, N., Pasalar, P., & Amanlou, M. (2014). A preliminary investigation of anticholinesterase activity of some Iranian medicinal plants commonly used in traditional medicine. DARU, Journal of Pharmaceutical Sciences, 22(1), 1-5. https://doi.org/10.1186/2008-2231-22-17/TABLES/1
12. Khanavi, M., Maadani, S., Farahanikia, B., Eftekhari, M., & Sharifzadeh, M. (2017). Effect of the methanolic extracts of different parts of Ferula assa-foetida on naloxone-induced withdrawal behavior in morphine-dependent mice. Avicenna Journal of Phytomedicine, 7(5), 426./pmc/articles/PMC5641417/
13. Koschel, J., Ray Chaudhuri, K., Tönges, L., Thiel, M., Raeder, V., & Jost, W. H. (2021). Implications of dopaminergic medication withdrawal in Parkinson's disease. Journal of Neural Transmission, 1, 1-10. https://doi.org/10.1007/S00702-021-02389-X/FIGURES/1
14. Krishna, R., Ali, M., & Moustafa, A. A. (2014). Effects of combined MAO-B inhibitors and levodopa vs monotherapy in Parkinson's disease. Frontiers in Aging Neuroscience, 6(Jul.), 180. https://doi.org/10.3389/FNAGI.2014.00180/BIBTEX
15. Lee, T. K., & Yankee, E. L. (2021). A review on Parkinson's disease treatment. Neuroimmunology and Neuroinflammation, 8, 222. https://doi.org/10.20517/2347-8659.2020.58
16. Maiti, P., Manna, J., Dunbar, G. L., Maiti, P., & Dunbar, G. L. (2017). Current understanding of the molecular mechanisms in Parkinson's disease: Targets for potential treatments. In Translational Neurodegeneration (Vol. 6, Issue 1, pp. 1-35). https://doi.org/10.1186/s40035-017-0099-z
17. MAO-B inhibitors (rasagiline, selegiline, safinamide)|Parkinson's UK. (n.d.). Retrieved Jun. 14, 2022, from https://www.parkinsons.org.uk/information-and-support/mao-b-inhibitors-rasagiline-selegiline-safinamide
18. Mhyre, T. R., Boyd, J. T., Hamill, R. W., & Maguire-Zeiss, K. A. (2012). Parkinson's disease. Subcellular Biochemistry, 65, 389-455. https://doi.org/10.1007/978-94-007-5416-4_16
19. Mohammadhosseini, M., Mahdavi, B., & Shahnama, M. (2015). Chemical Composition of Essential Oils from Aerial Parts of Ferula gummosa (Apiaceae) in Jajarm Region, Iran Using Traditional Hydrodistillation and Solvent-Free Microwave Extraction Methods: A Comparative Approach. Journal of Essential Oil-Bearing Plants, 18(6), 1321-1328. https://doi.org/10.1080/0972060X.2015.1024445
20. Mohammadhosseini, M., & Nekoei, M. (2014). Chemical Compositions of the Essential Oils and Volatile Compounds from the Aerial Parts of Ferula ovina Using Hydrodistillation, MAHD, SFME and HS-SPME Methods. Journal of Essential Oil—Bearing Plants, 17(5), 747-757. https://doi.org/10.1080/0972060X.2014.884951
21. Mohammadhosseini, M., Venditti, A., Sarker, S. D., Nahar, L., & Akbarzadeh, A. (2019). The genus Ferula: Ethnobotany, phytochemistry and bioactivities—A review. In Industrial Crops and Products (Vol. 129, pp. 350-394). Elsevier B. V. https://doi.org/10.1016/j.indcrop.2018.12.012
22. Müller, T. (2012). Drug therapy in patients with Parkinson's disease. Translational Neurodegeneration, 1(1), 1-13. https://doi.org/10.1186/2047-9158-1-10/TABLES/5
23. Nagatsua, T., & Sawadab, M. (2009). L-dopa therapy for Parkinson's disease: Past, present, and future. Parkinsonism & Related Disorders, 15(1), S3-S8. https://doi.org/10.1016/S1353-8020(09)70004-5
24. Rabinak, C. A., & Nirenberg, M. J. (2010). Dopamine Agonist Withdrawal Syndrome in Parkinson Disease. Archives of Neurology, 67(1), 58-63. https://doi.org/10.1001/ARCHNEUROL.2009.294
25. Riederer, P., & Laux, G. (2011). MAO-inhibitors in Parkinson's Disease. Experimental Neurobiology, 20(1), 1-17. https://doi.org/10.5607/EN.2011.20.1.1
26. Rizek, P., Kumar, N., & Jog, M. S. (2016). An update on the diagnosis and treatment of Parkinson disease. CMAJ, 188(16), 1157-1165. https://doi.org/10.1503/CMAJ.151179
27. Solla, P., Fasano, A., Cannas, A., & Marrosu, F. (2017). Dopamine agonist withdrawal syndrome in Parkinson's disease. Journal of the Neurological Sciences, 382, 47-48. https://doi.org/10.1016/j.jns.2017.08.3263
28. Solla, P., Fasano, A., Cannas, A., Mulas, C. S., Marrosu, M. G., Lang, A. E., & Marrosu, F. (2015). Dopamine agonist withdrawal syndrome (DAWS) symptoms in Parkinson's disease patients treated with levodopa-carbidopa intestinal gel infusion. Parkinsonism & Related Disorders, 21(8), 968-971. https://doi.org/10.1016/J.PARKRELDIS.2015.05.018
29. Tayeboon, G. S., Tavakoli, F., Hassani, S., Khanavi, M., Sabzevari, O., & Ostad, S. N. (2013). Effects of Cymbopogon citratus and Ferula assa-foetida extracts on glutamate-induced neurotoxicity. In Vitro Cellular and Developmental Biology—Animal, 49(9), 706-715. https://doi.org/10.1007/s11626-013-9656-7
30. Xing, Y., Li, N., Zhou, D., Chen, G., Jiao, K., Wang, W., Si, Y., & Hou, Y. (2017). Sesquiterpene Coumarins from Ferula sinkiangensis act as Neuroinflammation Inhibitors. Planta Medica, 83(01/02), 135-142. https://doi.org/10.1055/S-0042-109271
31. Yatham, P., Shukla, D., Srivastava, A. K., Pragadheesh, V. S., & Kumar, D. (2021). Purification and identification of anticancer organosulfides from Ferula assa-foetida gum: integrative analysis employing GC/GC-MS/RP-HPLC/NMR. Natural Product Research. https://doi.org/10.1080/14786419.2021.1922903
32. Zarmouh, N., Messeha, S., Elshami, F., & Soliman, K. (2016). Natural Products Screening for the Identification of Selective Monoamine Oxidase-B Inhibitors. European Journal of Medicinal Plants, 15(1), 1-16. https://doi.org/10.9734/ejmp/2016/26453

1. A composition comprising of sesquiterpene coumarins enriched fractions in fatty acid or fatty ester containing oil phase along with emulsifiers, co-surfactants, and water.

2. The composition as claimed in claim 1, wherein the sesquiterpene coumarins is having a general molecular formula C₂₄₋₂₆H₂₆₋₃₆O₃₋₆ and molecular weight in the range of 362-444.

3. The composition as claimed in claim 1, wherein the sesquiterpene coumarins are selected from ferukrin (0.05-42.31%), assafoetidanol A (0.013-1.17%) ligupersin A (0.31-36.29%) kamolonol (1.50-46.67%), 10′-acetoxy-11′-hydroxy umbelliprenin (1.52-2.86%) epi-conferdione (0.24-12.24%), karatavicinol (0.07-6.13%) kellerin (1.43-17.63%), ferocaulidin (0.19-2.23%), famesiferol A (0.13-14.31%), galbanic acid (0.05-52.56%), szowitsiacoumarin B (5.18-16.18%), farnesiferol B (0.81-10.08%) gummosin (2.58-4.03%), farnesiferol C (0.01-40.98%), szowitsiacoumarin A (0.28-7.01%) and methyl galbanate (0.70-10.77%).

4. The composition as claimed in claim 1, wherein the fatty acid or fatty ester containing oil phase is selected from but not limited to caprylic acid, isopropyl myristate, olive oil, coconut oil, and ethyl oleate either alone or in combinations.

5. The composition as claimed in claim 1, wherein the emulsifier is selected from the group consisting of non-ionic, cationic, and anionic emulsifiers.

6. The composition as claimed in claim 5, wherein the non-ionic emulsifier is Tween 20, Tween 60, Tween 80, Span 80, Span 20, Brij35.

7. The composition as claimed in claim 5, wherein the cationic emulsifier is dodecyl trimethyl ammonium chloride, cetyltrimethylammonium bromide.

8. The composition as claimed in claim 5, wherein the anionic emulsifier is sodium lauryl sulfate, trigol.

9. The composition as claimed in claim 1, wherein the co-surfactants are selected from propylene glycol and PEG400.

10. The composition as claimed in claim 1, for use in the treatment of Parkinson's disease.

11. A process for the preparation of the composition as claimed in claim 1, wherein the process comprising the steps of—
a. treating sesquiterpene coumarin enriched fraction with fatty acid or fatty ester containing oil phase in a ratio of 1:1-1:10 w/w with high or low external energy input for 5 min to 24 hours at 10° C. to 40° C. to obtain a clear transparent solution,
b. treating the solution as obtained in step (a) with non-ionic, cationic, or anionic emulsifiers with or without co-surfactants in the ratio of 1:1.1-1:12.5 w/w with high or low energy agitation for 1 min to 24 hours at 10° C. to 30° C. to obtain a resultant mixture,
c. dispersing the resultant mixture obtained in step (b) in water in the ratio of 1:0.5-1:20% w/w with agitation for 1 min to 24 hours to obtain the water-soluble composition.

12. A process of preparation of sesquiterpene coumarin enriched fractions as claimed in claim 11, wherein the process comprising the steps of:
(a) extracting Ferula oleo-gum-resin multiple times with organic solvents of polarity index −6 to obtain a extact,
(b) combining and concentrating the extract of step (a) to an ethyl-acetate free residue,
(c) separating the ethyl-acetate free residue of step (b) using silica gel followed by elution with hexane-chloroform and chloroform-methanol gradient to obtain the sesquiterpene coumarin enriched fraction.

13. A method of treating Parkinson's disease in a subject comprising administering to said subject a therapeutically effective amount of the water soluble composition of claim 1.

1-13. (canceled)

14. A composition comprising a sesquiterpene coumarin enriched fraction in a fatty acid or a fatty ester containing oil phase in combination with an emulsifier, a cosurfactant, and water.

15. The composition of claim 14, wherein the sesquiterpene coumarin has a general molecular formula C₂₄₋₂₆H₂₆₋₃₆O₃₋₆ and a molecular weight in the range of 362-444.

16. The composition of claim 14, wherein the sesquiterpene coumarin is selected from ferukrin (0.05-42.31%), assafoetidanol A (0.013-1.17%), ligupersin A (0.31-36.29%), kamolonol (1.50-46.67%), 10′-acetoxy-11′-hydroxy umbelliprenin (1.52-2.86%), epi-conferdione (0.24-12.24%), karatavicinol (0.07-6.13%), kellerin (1.43-17.63%) ferocaulidin (0.19-2.23%), famesiferol A (0.13-14.31%), galbanic acid (0.05-52.56%) szowitsiacoumarin B (5.18-16.18%), farnesiferol B (0.81-10.08%), gummosin (2.58-4.03%), farnesiferol C (0.01-40.98%), szowitsiacoumarin A (0.28-7.01%), and methyl galbanate (0.70-10.77%).

17. The composition of claim 14, wherein the fatty acid or fatty ester of the oil phase is selected from the group consisting of caprylic acid, isopropyl myristate, olive oil, coconut oil, and ethyl oleate, and combinations thereof.

18. The composition of claim 14, wherein the emulsifier is selected from the group consisting of non-ionic, cationic, and anionic emulsifiers.

19. The composition of claim 18, wherein the non-ionic emulsifier is selected from the group consisting of Tween 20, Tween 60, Tween 80, Span 80, Span 20, and Brij35.

20. The composition as claimed in claim 18, wherein the cationic emulsifier is dodecyl trimethyl ammonium chloride or cetyltrimethylammonium bromide.

21. The composition of claim 18, wherein the anionic emulsifier is sodium lauryl sulfate or trigol.

22. The composition of claim 14, wherein the co-surfactant is selected from propylene glycol and PEG400.

23. A method of treating a patient afflicted with Parkinson's disease, the method comprising administering to the patient an effective amount of the composition of claim 14.

24. A process for the preparation of the composition claim 14, the process comprising:
(a) treating sesquiterpene coumarin enriched fraction with fatty acid or fatty ester containing oil phase in a ratio of 1:1-1:10 w/w with high or low external energy input for 5 min to 24 hours at 10° C. to 40° C. to obtain a clear transparent solution,
(b) treating the solution as obtained in step (a) with non-ionic, cationic, or anionic emulsifiers with or without co-surfactants in the ratio of 1:1.1-1:12.5 w/w with high or low energy agitation for 1 min to 24 hours at 10° C. to 30° C. to obtain a resultant mixture, and
(c) dispersing the resultant mixture obtained in step (b) in water in the ratio of 1:0.5-1:20% w/w with agitation for 1 min to 24 hours to obtain the composition.

25. A process for the preparation of sesquiterpene coumarin enriched fraction according to claim 23, the process comprising:
(a) extracting Ferula oleo-gum-resin multiple times with an organic solvent of polarity index −6 to obtain an extract,
(b) combining and concentrating the extract of (a) to an ethyl-acetate free residue, and
(c) separating the ethyl-acetate free residue of (b) by utilizing a silica gel followed by eluting with hexane-chloroform and chloroform-methanol gradient to obtain the sesquiterpene coumarin enriched fraction.
